(19) United States
(12) Patent Application Publication
(10) Pub. No.: US 2026/0242323 A1
(43) Pub. Date: Aug. 20, 2026

(54) MANUFACTURING PROCESS OF METAL-ORGANIC FRAMEWORK POROUS MATERIAL OF ALUMINIUM AND TEREPHTHALIC ACID

(71) Applicant: CHIANG MAI UNIVERSITY, Mueang Chiang Mai (TH)

(72) Inventors: Saranphong YIMKLAN, Mueang Chiang Rai (TH); Yothin CHIMUPALA, Lampang (TH); Natthawat SEMAKUL, Lampang (TH); Chitsanupong PHROMMA, Phayao (TH); Nithiwat SAMMAWIPAWEKUL, Mueang Chiang Rai (TH)

(73) Assignee: CHIANG MAI UNIVERSITY, Mueang Chiang Mai (TH)

(21) Appl. No.: 19/164,583

(22) PCT Filed: Aug. 17, 2023
(86) PCT No.: PCT/TH2023/050022

§ 371 Date: Sep. 12, 2025

(30) Foreign Application Priority Data
Aug. 17, 2023 (TH) 2301005092

(51) Int. Cl. C07C 51/41

(52) U.S. Cl. CPC C07C 51/418

(57) ABSTRACT

A manufacturing process of metal-organic framework porous material of aluminium and terephthalic acid comprises the steps of aluminium substrate synthesis with elution of aluminium ions from the aluminium substrate using a basic solution followed by adjustment of pH using an acidic solution; terephthalic acid synthesis from polyethylene terephthalate (PET) by distillation method in a basic solution followed by adjustment of pH using an acidic solution to yield terephthalic acid precipitate, filtering, drying and dissolving of the obtained terephthalic acid in a basic solution until equivalent; addition of the aluminium substrate for a reaction; separation of the obtained precipitate of the metal-organic framework porous material product from solution; and drying.

### FIELD OF THE INVENTION

Science related to the manufacturing process of metal-organic framework porous materials of aluminium and terephthalic acid.

### BACKGROUND OF THE INVENTION

Metal-organic frameworks (MOF) are considered coordination polymers (CPs) with an attribute of net-like continuous framework and with important components being metal ions connected with organic ligands using coordinate covalent bonds via donor atoms, which come from inorganic substances'functional groups. Metal organic framework materials have the potential because of their large surface areas, high crystallinity, high porosity, low density, and tolerance to very high temperature; hence high efficiency in application to the chemical industrial sector, particularly as regards gas storage, separation of desired gases from impurities in manufacturing lines; as well as their catalysis capability.

Metal-organic framework synthesis can be performed using hydrothermal synthesis or solvothermal synthesis by mixing substances for synthesis together and putting in autoclave for heating under conditions having water or organic substances as solvents. During heating at preferred temperature, frameworks are formed. A form of metal-organic framework materials is aluminium-terephthalate metal-organic framework (MIL-53 (Al) or [Al(OH)(C₈H₄O₄)]ₙ type) consisting of an inorganic chain [Al—OH], which connects with additional four inorganic chains via terephthalate-based linker molecule. Each metal location harmonizes with one another via six oxygen atoms; four of which come from four different carboxylate groups, and the other two are in two different μ-OH groups.

Aluminium-terephthalate metal-organic frameworks (MIL-53 (Al) type) are desired in diverse chemical industries, hence manufacturing industries of aluminium-terephthalate metal-organic framework material (MIL-53 (Al) type) in the United Kingdom, including Promethean Particles with a manufacturing rate over 1,000 tonnes per annum, accounting for an income of around 500,000 million Baht per annum (516,078 Baht/kilogram). In addition, the said material under the tradename Basolite® A100 is manufactured by BASF (at a price of 281,292 Baht/500 grams). Aluminium-terephthalate metal-organic frameworks (MIL-53 (Al) type) are considered a highly porous material with specific surface areas greater than 2,000 square metres per gram, several-fold higher than zeolites widely used in the petrochemical industry in Thailand.

After performing patent database searches, some prior inventions relating to synthesis processes of aluminium metal-organic framework using an organic ligand being polyethylene terephthalate (PET) or terephthalic acid were found. For example:

A Chinese patent, publication number CN110283353B, with the invention title ‘Method for preparing metal organic skeleton material by step degradation of used polyester plastic’ describes a method for preparing metal organic framework material using polyester plastic from a group consisting of polyethylene terephthalate (PET), polybutylene terephthalate (PBT) or polytrimethylene terephthalate (PTT) as a substrate. Preparation steps consist of the addition of substrate of polyacid into sodium acetate buffer solution and stirring until the solution is clear. Then polyester waste is added, mixed well and heated at 170-190 degrees Celsius for 60-80 hours. Then it is left to cool down, filtered, rinsed and baked.

A Chinese patent, publication number CN108676174A, with the invention title ‘Method for preparing metal organic framework material from PET (polyethylene terephthalate) waste materials’ describes a process using polyethylene terephthalate waste (PET waste) for preparing metal organic framework material, where the steps consist of weighing zinc nitrate hexahydrate (Zn(NO3)2·6H2O) and polyethylene terephthalate waste at the ratio of 2:1-1:2; bringing them into a solvothermal reaction in a hydrothermal reactor at a temperature of 150 degrees Celsius for 24 hours; rinsing them, when the time is up, with N-methylpyrrolidone and methanol solvents, respectively; and then performing vacuum drying.

A United States patent, publication number US20120035365A1, with the invention title ‘Method for Manufacturing Porous Materials from Waste PET Bottle’ describes the manufacture of metal organic framework materials from waste PET bottles, where the steps consisting of filling a transition metal being zinc (Zn), an amine being pyridine or an alkyl polyamine, polyethylene terephthalate phosphate and water in a closed container; allowing a reaction at a temperature between 120-200 degrees Celsius under pressure between 1-100 atm for 48-168 hours; and then precipitating the obtained metal organic framework material.

A Chinese patent, publication number CN114106344A, with the invention title ‘Preparation method of aluminium metal organic framework material Al-MIL-53’ describes the preparation of aluminium substrate (Al-MIL-53) for making the metal organic framework material where the substrate is aluminium nitrate and/or aluminium chloride. A manufacturing process consists of a step of mixing a combined solvent, consisting of a solubilizer at 0.1-5% by weight, water at 49-90% by weight and dimethylformamide at 9-50% by weight, with the aluminium substrate and an organic ligand being terephthalic acid or 2-aminoterephthalic acid. The ratio by weight of the aluminium substrate per the organic ligand is 1 per 0.8-1.5, and the ratio by weight of the combined solvent per the aluminium substrate is 10-30 per 1. The reaction takes place at 100-250 degrees Celsius under autogenous pressure for 30-96 hours. The obtained solid is harvested, dried and calcined.

An Indian patent, publication number IN2397CHENP2008A, with the invention title ‘Porous metal-organic framework material comprising a further polymer’ describes a porous metal-organic framework material consisting of metal framework being a bidentate organic compound (such as terephthalic acid, isoterephthalic acid, 2,6-naphthalenedicarboxylic acid or 1,3,5-benzenetricarboxylic acid) connected with metal ions (such as magnesium, calcium, iron, zinc aluminium, nickel or copper) and a polymer (such as aromatic polymer, polyamide, polyester, polyether, polyacetal, polycarbonate or polyacrylate) for manufacturing the metal organic framework material with a gas-adsorbing property, such as for hydrogen, etc.

It can be seen that the above prior inventions have limitations in terms of product synthesis control, high energy consumption and use of organic solvents that are not environmentally friendly. In addition, manufacturing process yield percentages are low; the manufacture takes long time; and expensive substrates are used. This invention, however, focuses on the synthesis of aluminium-terephthalate metal-organic framework material (MIL-53 (Al) type) specifically using aluminium metal by using waste drink tins as aluminium source, waste polyethylene terephthalate (PET) bottles as terephthalic acid source, unsophisticated synthesis technique, a short period, environmentally friendly organic solvent and low energy, capable of controlling the pure phase formation and particle size and morphology.

### SUMMARY OF THE INVENTION

The manufacturing process of the metal-organic framework porous material of aluminium and terephthalic acid comprises steps of aluminium substrate synthesis, with elution of aluminium ions from the aluminium substrate using a basic solution followed by adjustment of pH between 3-7 using an acidic solution in a reactor; and terephthalic acid synthesis from polyethylene terephthalate (PET) by distillation method in a basic solution at the temperature of 50-70 degrees Celsius for 12-36 hours followed by adjustment of pH between 0-3 using an acidic solution in a reactor to yield terephthalic acid precipitate, filtering and drying by baking at the temperature of 70-90 degrees Celsius for 1-3 hours. Then the obtained terephthalic acid is dissolved in a basic solution until equivalent with the measured pH between 7-12. The aluminium substrate is added for a reaction of which temperature is between 25-120 degrees Celsius under the atmospheric pressure for a reaction period of 1-12 hours. Then the obtained precipitate of the metal-organic framework porous material is separated from solution and then dried by baking at the temperature of 70-90 degrees Celsius for 1-5 hours.

The goal of this invention is to provide the aluminium-terephthalate metal-organic framework material (MIL-53 (Al) type) using the unsophisticated manufacturing process, short period and low energy, capable of controlling the pure phase formation and particle size and morphology, helping to meet the industrial needs for aluminium-terephthalate metal-organic framework materials (MIL-53 (Al) type), adding values and solving a problem of in-house waste, creating investment opportunities in Thai and regional markets, and capable of creating an economic advantage for business operation, both in-house and abroad.

### BRIEF DESCRIPTION OF DRAWINGS

FIG. 1: terephthalic acid spectrum in a solvent (DMSO-d6) as measured with the proton-nuclear magnetic resonance (1H-NMR) technique

FIG. 2: X-ray diffraction pattern of the prepared aluminium-terephthalate metal-organic framework material (MIL-53 (Al) type) at a production capacity of 0.1 kilogram

### DETAILED DESCRIPTION OF THE INVENTION

The manufacturing process of the metal-organic framework porous material of aluminium and terephthalic acid comprises the following steps.

A. Synthesise the aluminium substrate, with elution of aluminium ions from the aluminium substrate using a basic solution followed by adjustment of pH between 3-7 using an acidic solution in a reactor.

In an embodiment, the aluminium substrate can be selected from any one of waste aluminium tins, aluminium ingot, aluminium halide compounds, aluminium sulphate compounds, aluminium nitrate compounds, aluminium acetate compounds, aluminium perchlorate compounds, slag or combination thereof.

In an embodiment, the basic solution can be selected from lithium hydroxide, sodium hydroxide, potassium hydroxide, barium hydroxide, calcium hydroxide, ammonium hydroxide or combination thereof.

In another embodiment, the acidic solution can be selected from acetic acid, hydrochloric acid, sulphuric acid, nitric acid or combination thereof.

In another embodiment, the ratio of the aluminium substrate per the acidic or basic solution is 1 per 1-10.

B. Synthesise terephthalic acid from polyethylene terephthalate (PET) by distillation method in a basic solution at the temperature of 50-70 degrees Celsius for 12-36 hours. Then adjust pH between 0-3 using an acidic solution in a reactor to yield terephthalic acid precipitate, filter and dry by baking at the temperature of 70-90 degrees Celsius for 1-3 hours.

In another embodiment, the polyethylene terephthalate plastic can be selected from waste polyethylene terephthalate plastic, polyethylene terephthalate pellets, waste of polyethylene terephthalate industries or combination thereof.

In another embodiment, the basic solution can be selected from any one of lithium hydroxide, sodium hydroxide, potassium hydroxide, barium hydroxide, calcium hydroxide or ammonium hydroxide in water or alcoholic solvent, or combination thereof.

In another embodiment, the ratio of polyethylene terephthalate per the basic solution is 1 per 0.25-4.0.

In another embodiment, the basic solution has the weight between 5-50 times of the total weight of the substrate.

In another embodiment, the acidic solution can be selected from acetic acid, hydrochloric acid, sulphuric acid, nitric acid or combination thereof.

C. Allow a reaction for manufacturing the metal-organic framework porous material of aluminium and terephthalic acid by dissolving terephthalic acid obtained from step B in a basic solution until equivalent with the measured pH between 7-12. Add the aluminium substrate obtained from step A for the reaction of which temperature is between 25-120 degrees Celsius under the atmospheric pressure for a reaction period of 1-12 hours.

In another embodiment, the basic solution can be selected from any one of lithium hydroxide, sodium hydroxide, potassium hydroxide, barium hydroxide, calcium hydroxide, ammonium hydroxide or combination thereof.

In another embodiment, a ratio by weight of the aluminium substrate per terephthalic acid is 1 per 1.

In another embodiment, the pH in the reaction between the aluminium substrate and terephthalic acid is between 3-4.

D. Separate precipitate of the metal-organic framework porous material product obtained from the reaction in step C from solution and then dry by baking at the temperature of 70-90 degrees Celsius for 1-5 hours.

In another embodiment, step C includes reaction pH adjustment to 7 at the ending of the reaction prior to filtering the obtained precipitate of the metal-organic framework porous material product.

In another embodiment, the reactor is a parallel reactor or a wet reactor.

The aluminium-terephthalate metal-organic framework (MIL-53 (Al) type) manufactured from the process according to this invention has the purity higher than 90%, metal impurities less than 1% by weight, particle size distribution range less than 50 micrometres and a surface area greater than 500 square metres/gram (m2/g).

Hereinafter, description of the invention will be by exemplifying the invention to facilitate a clearer understanding. However, the manufacturing process of metal-organic framework porous material of aluminium and terephthalic acid and related information to follow merely are examples with an objective to demonstrate for better understanding of the invention and does not mean to limiting invention results to those in the examples.

### EXAMPLE 1: ANALYTICAL RESULTS OF OBTAINED TEREPHTHALIC ACID CHARACTERISTICS

Polyethylene terephthalate (PET) bottles were digested by the distillation method in a basic solution to yield terephthalic acid, and the manufacturing process of the aluminium-terephthalate metal-organic framework material (MIL-53 (Al) type) at a production capacity of 0.1 kilogram per production cycle was tested.

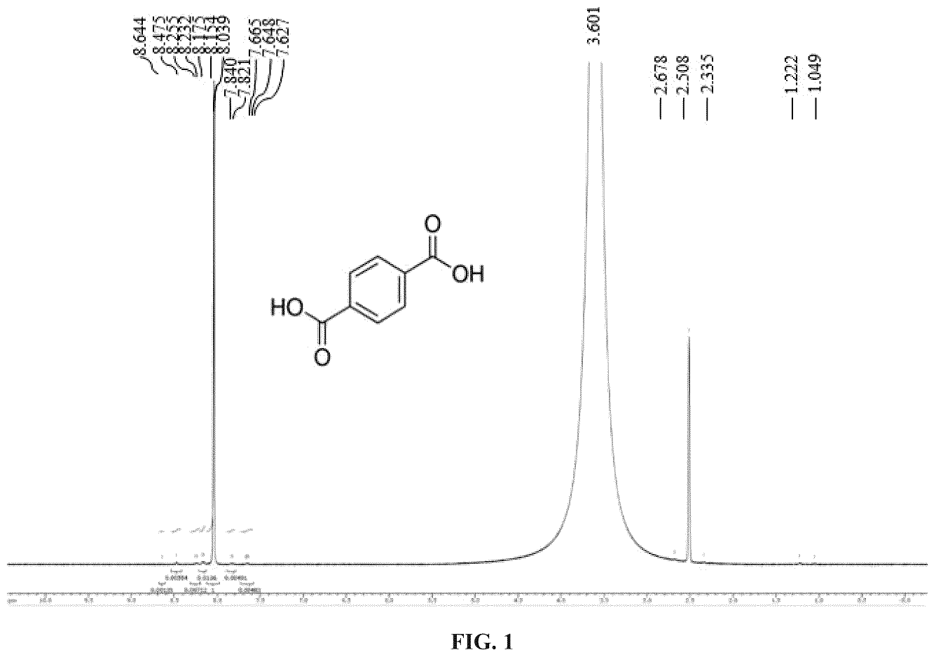

Analytical results of the obtained terephthalic acid characteristics by the proton-nuclear magnetic resonance (1H-NMR) technique showed a single signal at 8.039 ppm representing the 4 hydrogen locations of terephthalic acid where the 4 hydrogen locations were structurally equivalent because of the equal space from the carboxylate group (—COO—), thereby confirming the terephthalic acid identification, and other impurity signals in a range of 7.627-8.644 ppm. Upon calculation of terephthalic acid and impurity percentages, it was found that terephthalic acid proportion was as high as 99%. It can be concluded that the synthesised terephthalic acid has sufficiently high purity for the manufacture of the aluminium-terephthalate metal-organic framework material (MIL-53 (Al) type) (FIG. 1).

### EXAMPLE 2: ANALYTICAL RESULTS OF MANUFACTURED ALUMINIUM-TEREPHTHALATE METAL-ORGANIC FRAMEWORK MATERIAL (MIL-53 (AL) TYPE)

Characteristics of the aluminium-terephthalate metal-organic framework material (MIL-53 (Al) type) prepared at a production capacity of 0.1 kilogram per production cycle were analysed by the powder X-ray diffraction technique. It was found that the manufacturing process used was capable of preparing the aluminium-terephthalate metal-organic framework material (MIL-53 (Al) type) with high crystalline phase purity.

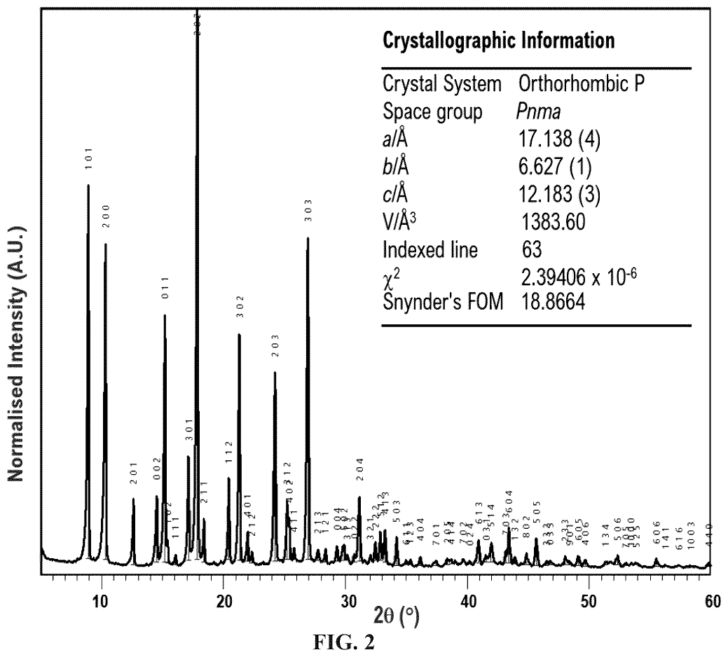

Crystalline structure of the prepared aluminium-terephthalate metal-organic framework material (MIL-53 (Al) type) at the production capacity of 0.1 kilogram was checked by the powder X-ray diffraction technique. Upon refinement compared with lattice parameters, atom locations and types in cell units of the aluminium-terephthalate metal-organic framework material (MIL-53 (Al) type) from the Crystallographic Information File (CIF) number CCDC 220475, reports in Cambridge Structural Database, phase identification and specification of Miller index by X'Pert High Score Plus, all peaks were found to be important. The prepared aluminium-terephthalate metal-organic framework material (MIL-53 (Al) type) had the 2θ location corresponding to peak of aluminium-terephthalate metal-organic framework material (MIL-53 (Al) type) in the database. It, therefore, was possible to specify that the prepared aluminium-terephthalate metal-organic framework material (MIL-53 (Al) type) had the crystalline structure identical to aluminium-terephthalate metal-organic framework material (MIL-53 (Al) type) with an orthorhombic crystal system; Pnma space group; cell unit parameters as follows: a=17.110(4) Å, b=6.615(2) Å, and c=12.171(3) Å; and cell unit volume=1377.69 Å3 (FIG. 2).

### BEST MODE OF THE INVENTION

As described in Detailed Description of the Invention

What is claimed is:

1. A manufacturing process of metal-organic framework porous material of aluminium and terephthalic acid, comprising the following steps.
A. Synthesise aluminium substrate, with elution of aluminium ions from the aluminium substrate using a basic solution followed by adjustment of pH between 3-7 using an acidic solution in a reactor.
B. Synthesise terephthalic acid from polyethylene terephthalate (PET) by distillation method in a basic solution at the temperature of 50-70 degrees Celsius for 12-36 hours. Then adjust pH between 0-3 using an acidic solution in a reactor to yield terephthalic acid precipitate, filter and dry by baking at the temperature of 70-90 degrees Celsius for 1-3 hours.
C. Allow a reaction for manufacturing the metal-organic framework porous material of aluminium and terephthalic acid by dissolving terephthalic acid obtained from step B in a basic solution until equivalent with the measured pH between 7-12. Add the aluminium substrate obtained from step A for the reaction of which temperature is between 25-120 degrees Celsius under the atmospheric pressure for a reaction period of 1-12 hours.
D. Separate precipitate of the metal-organic framework porous material product obtained from the reaction in step C from solution, and then dry by baking at the temperature of 70-90 degrees Celsius for 1-5 hours.

2. The manufacturing process of metal-organic framework porous material of aluminium and terephthalic acid according to claim 1, wherein the aluminium substrate in step A can be selected from any one of waste aluminium tins, aluminium ingot, aluminium halide compounds, aluminium sulphate compounds, aluminium nitrate compounds, aluminium acetate compounds, aluminium perchlorate compounds, slag, or combination thereof.

3. The manufacturing process of metal-organic framework porous material of aluminium and terephthalic acid according to claim 1, wherein the basic solution in step A can be selected from any one of lithium hydroxide, sodium hydroxide, potassium hydroxide, barium hydroxide, calcium hydroxide, ammonium hydroxide or combination thereof.

4. The manufacturing process of metal-organic framework porous material of aluminium and terephthalic acid according to claim 1, wherein the acidic solution in step A can be selected from any one of acetic acid, hydrochloric acid, sulphuric acid, nitric acid or combination thereof.

5. The manufacturing process of metal-organic framework porous material of aluminium and terephthalic acid according to claim 1, wherein the ratio of the aluminium substrate per the acidic or basic solution in step A is 1 per 1-10.

6. The manufacturing process of metal-organic framework porous material of aluminium and terephthalic acid according to claim 1, wherein the polyethylene terephthalate plastic in step B can be selected from any one of waste polyethylene terephthalate plastic, polyethylene terephthalate pellets, waste of polyethylene terephthalate industries or combination thereof.

7. The manufacturing process of metal-organic framework porous material of aluminium and terephthalic acid according to claim 1, wherein the basic solution in step B can be selected from any one of lithium hydroxide, sodium hydroxide, potassium hydroxide, barium hydroxide, calcium hydroxide or ammonium hydroxide in water or alcoholic solvent, or combination thereof.

8. The manufacturing process of metal-organic framework porous material of aluminium and terephthalic acid according to claim 1, wherein the ratio of polyethylene terephthalate per the basic solution is 1 per 0.25-4.0.

9. The manufacturing process of metal-organic framework porous material of aluminium and terephthalic acid according to claim 1, wherein the basic solution in step B has the weight between 5-50 times of the total weight of the substrate.

10. The manufacturing process of metal-organic framework porous material of aluminium and terephthalic acid according to claim 1, wherein the acidic solution in step B can be selected from any one of acetic acid, hydrochloric acid, sulphuric acid, nitric acid or combination thereof.

11. The manufacturing process of metal-organic framework porous material of aluminium and terephthalic acid according to claim 1, wherein the basic solution in step C can be selected from any one of lithium hydroxide, sodium hydroxide, potassium hydroxide, barium hydroxide, calcium hydroxide, ammonium hydroxide or combination thereof.

12. The manufacturing process of metal-organic framework porous material of aluminium and terephthalic acid according to claim 1, wherein a ratio by weight of the aluminium substrate per terephthalic acid in step C is 1 per 1.

13. The manufacturing process of metal-organic framework porous material of aluminium and terephthalic acid according to claim 1, wherein the pH of the reaction between the aluminium substrate and terephthalic acid in step C is 3-4.

14. The manufacturing process of metal-organic framework porous material of aluminium and terephthalic acid according to claim 1, wherein step C includes reaction pH adjustment to 7 at the ending of the reaction prior to filtering the obtained precipitate of the metal-organic framework porous material product.

15. The manufacturing process of metal-organic framework porous material of aluminium and terephthalic acid according to claim 1, wherein the reactor is a parallel reactor or a wet reactor.
